(19) United States
(12) Patent Application Publication
(10) Pub. No.: US 2016/0184161 A1
(43) Pub. Date: Jun. 30, 2016

(54) Open-Frame Leg Support For Colonoscopy

(71) Applicant: Mark Lee Appler, Mount Airy, NC (US)

(72) Inventor: Mark Lee Appler, Mount Airy, NC (US)

(21) Appl. No.: 14/583,581

(22) Filed: Dec. 26, 2014

(51) Int. Cl. A61G 13/12; A61B 1/31

(52) U.S. Cl. CPC A61G 13/1245; A61B 1/31

(57) ABSTRACT

An open-framed leg support for patients undergoing a colonoscopy that reduces intrusion into a physician's working space. The leg support has a sturdy base that rests on an examination table (10) and (11). It also has a support surface (23a) and (23b) upon which a patient's leg rests, and which is mechanically biased to prevent the leg from falling off of the support. Finally, it has a frame (14) and (15) that joins the base and the support surface on only one side, leaving the other sides open and free of encumbrances. Additionally, it has a crossbar (16) in the frame for stability, and a strut (20) connecting the crossbar to the leg support surface for additional support.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

1. Field of Invention

This invention relates to medical leg supports, and in particular, a leg support for a patient undergoing a colonoscopy.

2. Prior Art

A colonoscopy is a medical procedure entailing a physician's examination of a patient's large bowel and the distal portion of the patient's small bowel using a fiber-optic scope. It is an important procedure for diagnosis and treatment of a variety of gastrointestinal diseases. During the course of the colonoscopy, the patient is repositioned so that the physician may guide the scope through the patient's bowel. One position the patient may assume is lying on her back to facilitate abdominal counter-pressure or the use of fluoroscopy. If the patient has been heavily sedated, it is necessary to employ a device to maintain the patient's leg in an elevated position and keep it in place so that the physician continues to have adequate access to the patient's rectum.

The prior art in leg supports for patients undergoing colonoscopies was a firm foam cushion having a shape roughly similar to that of a cube, with a top, leg-contact surface, slightly smaller than the base, and having a wedge-shaped indent in which the leg may sit. However, this solid foam structure occupied a significant amount of space in the physician's working area above the examination table, beneath the leg it supported. Because of the size of the leg support, the physician had less space in which to maneuver the scope and guide it into the patient's body.

Another traditional option was to have an assistant hold the patient's leg. This required the presence of another person in the endoscopy suite, ultimately adding to the cost of the procedure.

My invention solves this problem by doing away with the solid form of a leg support, as well as the additional assistant's presence, and adopting a frame structure that is entirely open on three sides. Such a structure leaves a great deal of space free above the examination table in which the physician performing a colonoscopy may maneuver the scope.

### BACKGROUND OF THE INVENTION

### Objects and Advantages

The primary object of my invention is to provide support to a sedated patient's leg during the portion of a colonoscopy in which one leg must be suspended. The primary advantage of my invention is its open frame structure. This structure does not impede the physician's maneuverability as the prior art did. Instead, it leaves plenty of space beneath the patient's leg in which the physician may operate and guide the scope. My invention also obviates the cost of having an additional assistant to hold the patient's leg.

Further objects and advantages will become apparent from a consideration of the ensuing description and drawings.

### SUMMARY

My invention is a leg support for patients undergoing a colonoscopy. It comprises a surface upon which a patient's leg may rest, and which is mechanically biased to prevent the leg from falling off of the leg support, a base to rest the leg support upon an examination table, and a vertical frame connecting the two. The frame connects the surface to the base on only one side of the leg support, leaving the other three sides open. This open-framed structure gives a physician more working room on the examination table than prior leg supports did, and eliminates the need for another assistant.

### DRAWINGS

### Figures

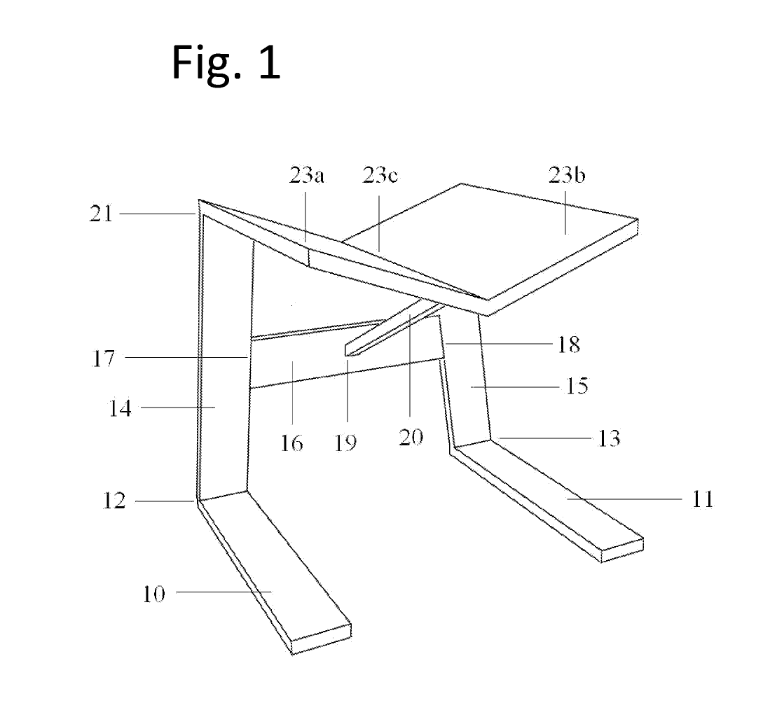

FIG. 1 is a perspective (front-upper) view of my invention's preferred embodiment.

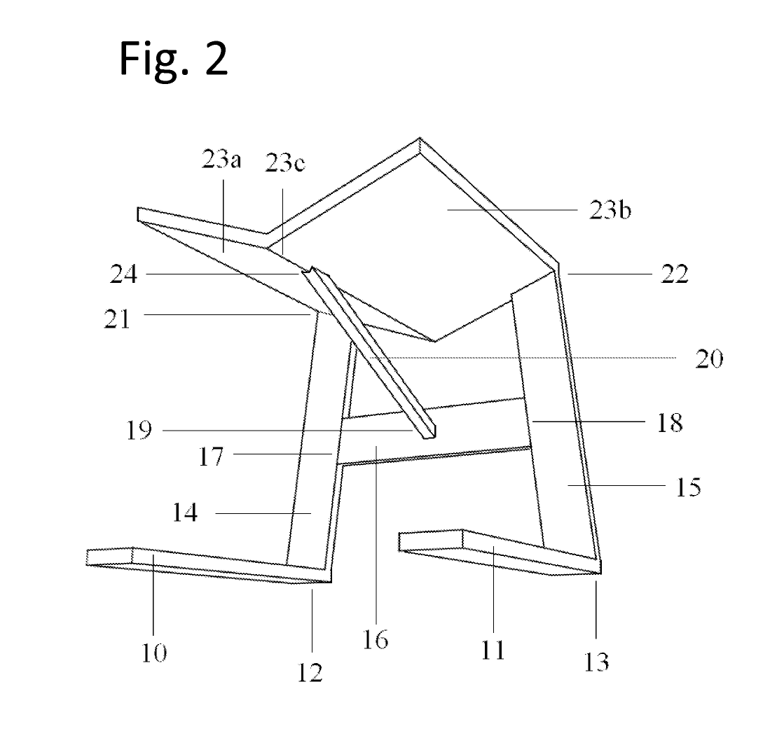

FIG. 2 is a perspective (front-lower) view of my invention's preferred embodiment.

### DRAWINGS—REFERENCE NUMERALS

10 base
11 base
12 base-frame weld
13 base-frame weld
14 frame
15 frame
16 crossbar
17 frame-crossbar weld
18 frame-crossbar weld
19 crossbar-strut weld
20 strut
21 frame-surface weld
22 frame-surface weld
23a leg support surface side
23b leg support surface side
23c leg support surface bend line
24 strut-surface weld

### DETAILED DESCRIPTION—FIGS. 1, 2

### Preferred Embodiment

A preferred embodiment of the support is illustrated in FIGS. 1 and 2.

The support comprises a pair of identical lateral bases 10 and 11 running parallel from front to back. These bases are preferably fashioned from bars of stainless steel.

Bases 10 and 11 are attached at base-frame welds 12 and 13, respectively, to frames 14 and 15, respectively. Each of said frames preferably consists of a bar of stainless steel. Frames 14 and 15 extend upward at an angle 90 degrees from longitudinal bases 10 and 11. Frames 14 and 15 are angled slightly inward toward the median.

Partway up frames 14 and 15 is a horizontal crossbar 16, joining the two frames. The crossbar connects to frames 14 and 15 at frame-crossbar welds 17 and 18, respectively. The crossbar preferably consists of a bar of stainless steel.

At the top of frames 14 and 15 is a leg support surface. In the preferred embodiment, this surface comprises a sheet of stainless steel that is bent at a line along the median 23c, leaving two upward-angled sides 23a and 23b that form a V-shaped frontal cross section. Leg support surface sides 23a and 23b are welded at the rear lateral corners to frames 14 and 15, respectively, by frame-surface welds 21 and 22, respectively.

Beneath the leg support surface is a strut 20. Said strut originates at the median of the anterior face of crossbar 16. Said strut connects to the crossbar at crossbar-strut weld 19. Said strut follows the median, and extends anteriorly at an upward angle, terminating at strut-surface weld 24, where it meets surface sides 23a and 23b at a point along surface bend line 23c. The strut preferably consists of a stainless steel bar.

### Operation—Preferred Embodiment

During a colonoscopy, many physicians will reposition a patient from a position of lying on her side to on her back once the fiber-optic scope has reached the patient's sigmoid colon. At this point, the patient's leg must be elevated so that it does not hamper the physician's access to the patient's rectum. A member of the medical team would then place the base of my invention 10 and 11 on the table upon which the patient was laying, with the front of my invention oriented toward the patient's body. The patient's leg that is closest to the physician would then be placed upon the leg support. Typically her calf would be cradled between leg support surface sides 23a and 23b, with the patient's foot toward the back of my invention. The weight of the patient's leg is supported by the frame 14 and 15, and further supported by the strut 20 and the crossbar 16.

During this phase of the colonoscopy, the physician is able to move his hands, the scope, and any other equipment freely in the space above the base 10 and 11, and beneath the leg support surface 23a and 23b and strut 20.

### CONCLUSIONS, RAMIFICATIONS, AND SCOPE OF INVENTION

The reader will see that my invention is a sturdy leg support that gives a physician performing a colonoscopy ample space in which to work.

While the above description contains specificities, these should not be understood as limitations on the scope of the invention, but as exemplifications of the presently preferred embodiment thereof. Other ramifications and variations are possible within the teachings of the invention. For example, the invention may be composed of other materials, such as an aluminum alloy or high-strength plastic. The invention may also be constructed with minor variations in form, such as a U-shaped leg support surface.

Thus the scope of the invention should be determined by the appended claims and their legal equivalents rather than by the examples given above.

I claim:

1. A leg support comprising:
a. in combination, a surface of sufficient size to support a leg of a human and a means for preventing said leg from becoming displaced from said surface,
b. a vertical frame supporting said surface and the means for preventing displacement of said leg, said vertical frame connecting to a single side of said surface so as to leave space beneath the remaining sides open and unencumbered,
c. a horizontal base underlying said vertical frame,
d. a means for joining said surface to said vertical frame, and
e. a means for joining said vertical frame to said horizontal base,
whereby a person could maneuver equipment in the space between said surface and said horizontal base.

2. The leg support recited in claim 1, wherein
the means for preventing displacement of said leg comprises
two planes of material, meeting at an angle.

3. The leg support recited in claim 1, wherein
the means for preventing displacement of said leg comprises a sheet of material curving downward, having a U-shaped cross-section.

4. The leg support recited in claim 1, wherein
said surface is covered by a soft material in order to reduce discomfort to said leg.

5. The leg support recited in claim 1, wherein
said surface is braced by at least one diagonal strut connecting said surface to said vertical frame.
